(19) United States
(12) Patent Application Publication
(10) Pub. No.: US 2016/0183912 A1
(43) Pub. Date: Jun. 30, 2016

(54) Apparatus and Method for Forward Facing Piezoelectric Element Ultrasound Stylus

(71) Applicant: UNIVERSITY OF IOWA RESEARCH FOUNDATION, Iowa City, IA (US)

(72) Inventor: Steven Richard Mickelsen, Iowa City, IA (US)

(21) Appl. No.: 14/847,509

(22) Filed: Sep. 8, 2015

Related U.S. Application Data
(60) Provisional application No. 62/047,336, filed on Sep. 8, 2014.

(51) Int. Cl. A61B 8/08

(52) U.S. Cl. CPC A61B 8/0841; A61B 8/488; A61B 2090/3788

(57) ABSTRACT

An apparatus and method for fabricating a forward facing piezoelectric stylus is provided. In a representative device, a piezoelectric element is connected to a stylus shaft, wherein the stylus shaft provides connectivity from the piezoelectric element to a console device. The stylus have shape such that multiple can be fabricated simultaneously in certain steps. In some instances, the stylus is adapted such that it can be placed within a needle, allowing easier placement of the needle.

### CLAIM OF PRIORITY

This application claims priority from U.S. Provisional Patent Application No. 62/047,336 filed on Sep. 8, 2014, which is relied upon and incorporated herein in its entirety by reference.

### BACKGROUND OF THE INVENTION

1. Technical Field

The present invention is in the technical field of medical sensing. More particularly, the present invention is in the field of ultrasound styli. The invention further relates to apparatus and methods to assist in the accurate placement of an object such as a needle into a patient.

2. Related Art

Medical practice often entails insertion of objects, such as needles or catheters, into patients, often in locations with little tolerance for error. Vascular access is the number one cause of complications in catheter based medical procedures.

Placement in the desired location may require several attempts, with each attempt increasing the risk of injury to critical structures. Even after multiple attempts, the tip of the needle may end up outside the target tissue entirely. Multiple passes and misplacement may also cause blockage, which itself requires yet another attempt to replace the blocked needle. Other complications of needle placement include infection and bleeding, so minimizing the number of times a needle must be placed is highly desirable.

Imaging techniques such as ultrasound guided percutaneous puncture provides some advantages over standard techniques that rely on anatomic landmarks and tactile feedback (palpating a pulse, for example). The operator holds an ultrasound probe in one hand and directs a needle with the other while observing changes in the tissue movement and needle shaft positioning. However, the ultrasound-guided technique requires the operator to give up tactile feedback from one hand to manage the probe. Ultrasound also may mislead the operator because it does not necessarily distinguish between needle body vs. needle tip positioning with respect to the target tissue/vessel.

A line of sight ultrasound stylus is particularly useful in percutaneous access to the pericardial space. The technical challenges of directing a needle into the normal pericardium without inadvertently hitting liver, lung, or small arterial blood vessels in the abdominal wall and/or diaphragm is substantial. The needle may be introduced through the parietal pericardium without advancing too far, risking puncture of the right ventricle. It is therefore important to know the relationship between the needle tip and the pericardial space on the millimeter scale.

While the basic concept of a forward facing single element ultrasonic probe was first patented in the 1980s, the focus on the industry has shifted to high-density 2D and 3D ultrasonic arrays. The clinical need for a simple forward facing imaging transducer for needle positioning remains and has not been answered despite substantial improvements in materials science, computing power, and advances in manufacturing. The challenges of visualizing the path of a needle will take can be addressed by placing a transducer in the tip of the shaft. However, there is the added challenge of integrating that concept into a collective technology that truly enables the operator to integrate this clinically needed concept into practice.

### SUMMARY OF INVENTION

In view of the foregoing problems, a sensing stylus in accordance with the present invention was made. The present invention relates to a device that overcomes these difficulties by providing a probe, such as an ultrasound imager, affording sensing of surrounding tissue. The sensing stylus is placed within the needle or catheter during insertion. The sensing stylus can be used to guide the needle or catheter into the desired position. Once the needle or catheter is in place, the stylus can be removed to provide access to the tissue. The present invention further relates to a method and apparatus to use imaging guidance to assist in placing an object such as a needle or a catheter into a body, thereby reducing the incidence of complications.

According to one embodiment of the invention, the apparatus includes a small diameter stylus with a piezoelectric element(s) fixed to the tip. The stylus is inserted into any standard percutaneous access needle and may be secured to the hub so that the distal tip of the piezoelectric element is aligned with the needle bevel opening. The length of the stylus may be complementary to a variety of standard access needle lengths (generally <20 cm). The diameter of the stylus may be constructed so that the outer diameter is less than the inner diameter of the chosen needle (generally <2 mm).

According to an embodiment of the invention, the stylus length and inner lumen size may be variable but is complementary/compatible to the intended standard hypodermic needles in clinical use. The stylus may be relatively rigid and could be made of any appropriate biocompatible material that can be sterilized. The stylus may be delivered to a sterile field in the standard fashion.

The present invention relates to a device that may be inserted into the proximal end of the standard hypodermic needle and secured with the lure lock hub. The transceiver cable may be connected to the controller/console by a cable or wireless transmission. The needle may be irrigated with saline or other appropriate solution using a fluid hub on the stylus and may be connected to IV lines of standard fluid manifold.

The present invention further relates performing medical procedures using the standard needle and procedural technique. Once the percutaneous needle is inserted through the dermal layer, the piezoelectric transceiver may automatically provide an M-mode and/or Doppler color image of the path directly in front of the needle. The piezoelectric transceiver may also be configured to deliver destructive sonographic energy for the purpose of soft tissue ablation and/or disintegration of mineralized anatomic structures. The practitioner can use the additional imaging data to identify the best needle path and avoid unintentional puncture of collateral anatomy. Once the needle has reached the objective position of the medical procedure, the stylus can be removed. According to an embodiment of the invention, the apparatus and methods provide a means to gain additional anatomic information without substantially changing the workflow of common percutaneous techniques used in clinical practice.

The present invention further relates to an article of manufacture that enables the practitioner to observe tissue structures in the line of sight directly ahead of the needle path. In an embodiment, the device allows for performing M-mode ultrasound with color Doppler using a single element piezoelectric transceiver. This imaging modality can provide reliable temporal and spatial data in real time as the needle is advanced through the tissues. The linear path from percutaneous entry site to the heart or other organ/tissue can be visualized once the probe is first inserted under the skin. An ultrasound image from the needle tip can provide critical anatomic and geometric information for procedural adaptations and planning before the needle is advanced further. Fine adjustments can be made to avoid collateral structures and allow the operator to reliably know when the needle tip is in contact with a structure, such as the pericardium. The line of sight ultrasound stylus device may be connected to a console to display real-time M-mode ultrasound and/or Doppler data. The stylus piezoelectric transceiver may primarily be used to display M-mode sonographic data as the needle is moved through tissues; color Doppler data can be added as desired to help identify small branch vessels and target anatomy. The display shows the operator what lies directly in front of the needle and frees the operator's hand so they can perform the percutaneous stick using familiar tactile feedback and anatomic landmarks.

In an aspect according to an embodiment of the invention, a fluid lumen provides a means to make injections, aspirate body fluid, and/or transduce pressure measurements in the clinical setting. Once needle positioning is achieved the stylus can be removed opening the needle lumen fully. The aspect may be useful in a number of standard clinical applications ranging from joint aspiration, lumbar puncture, vascular access, paracentesis, thoracentesis, pericardiocentesis, and percutaneous pericardial access. In an embodiment, the device further provides for the injection of fluid and/or radiopaque contrast under direct ultrasound visualization (in concert with other imaging modalities such as fluoroscope), facilitating very fine adjustments as the needle tip is advanced. The line of sight ultrasound stylus can be left in place until pericardial access is confirmed. This enables a more reliable atraumatic pericardial access procedure further modifying the percutaneous Seldinger access technique commonly used for subxiphoid approach.

### BRIEF DESCRIPTION OF THE DRAWINGS

FIG. 1A is a schematic view of a sensing stylus according to an embodiment of the invention.

FIG. 1B shows details of the stylus in its operation position inside the standard needle at the distal tip, according to an embodiment of the invention.

FIG. 1C illustrates details of the stylus hub connected to the needle hub via a lure lock connection, according to an embodiment of the invention.

FIG. 1D illustrates the placement of a stylus into a needle, according to an embodiment of the invention.

FIG. 1E shows the back side of the stylus hub, according to an embodiment of the invention.

FIG. 1F shows the front side of the stylus hub, according to an embodiment of the invention.

FIG. 2 illustrates the interface of a portable tablet-sized electromechanical device with the stylus, according to an embodiment of the invention.

FIG. 3 illustrates steps that may be performed to use the device, according to an embodiment of the invention.

FIG. 4 illustrates steps that may be performed to use the device, according to an embodiment of the invention.

FIG. 5 shows a collection of fabricated styli, according to an embodiment of the invention.

FIG. 6 illustrates a collection of fabricated styli having a polygon short-axis shape and conductor core, according to an embodiment of the invention.

FIG. 7 illustrates the construction characteristics of a stylus, according to an embodiment of the invention.

### DETAILED DESCRIPTION OF THE EMBODIMENTS

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

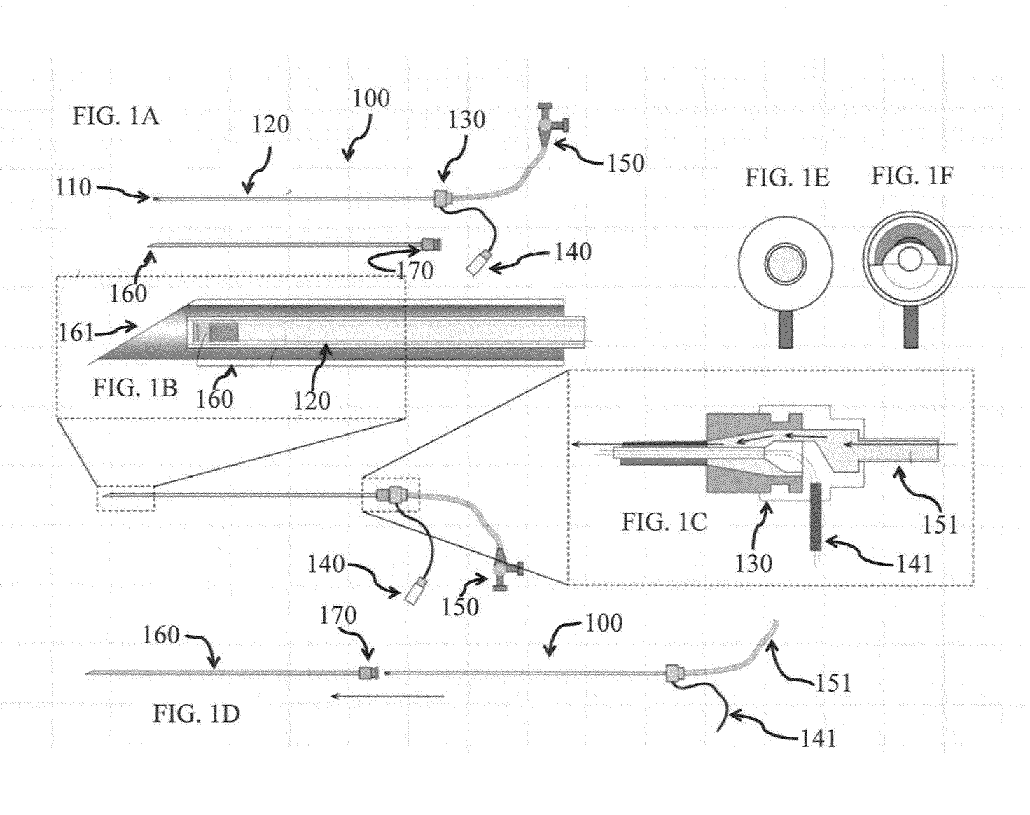

FIG. 1 illustrates a diagram depicting a piezoelectric element on the tip of a semi rigid stylus with connecting wire and a standard percutaneous needle. A piezoelectric element 110 is situated at the end of the shaft 120 of the stylus. A first lure lock stopcock 130 with a short connecting tube is connected to the end of the shaft 120 of the stylus and has a controller port 141 and a fluid port 151. An ultrasound controller wire connector 140 connects to the controller port 141 of the lure lock stopcock 130. A second lure lock stopcock 150 with a short connecting tube is further connected to the fluid port 151 of the first lure lock stopcock 130 with a short connecting tube.

The piezoelectric element 110 located at the distal tip may have its polar alignment parallel to the long axis of the stylus. The stylus may have a lens, matching layer, piezoelectric element(s), backing material, electrodes, and conductors integrated into the fabrication process, according to an embodiment. The stylus may be integrated into a lure lock cap that can connect to a standard needle lure lock hub 170.

A needle 160 for percutaneous access is provided. The needle may, in an embodiment, be a hypodermic type needle. The stylus shaft 120 may be inserted into the needle 160. The needle 160 may be terminated at one end with a hub 170 with a lure lock connection. In one embodiment, the length of the stylus may match the lumen length of the hypodermic needle 160 so that the piezoelectric element is aligned with the bevel of the distal needle opening. This length will vary for each specific application, with typical lengths of 3 to 10 cm. The diameter of the stylus will, according to an embodiment, be less than the minimum inner diameter of the needle 160 so that the stylus is arranged centrally with a small annulus of space between the stylus outer diameter and needed inner diameter. The annular space provides a conduit for fluid through the needle lumen.

The stylus hub may provide a watertight connection and a side arm with a lure lock connector 130 such that irrigation can be injected through the hub and into the inner lumen while the stylus 100 is in place. The stylus 100 may also have a highly flexible cable extending from the hub in an ergonomic position.

The stylus connector cable 140 may vary in length, and in one embodiment is 1-1.5M in length. The stylus connector cable 140 may be RFID (radio frequency identification) compatible so the console can identify the device. Multiple variations of the stylus 100 may be produced to accommodate different types of needle based procedural needs. The default settings may be adjusted accordingly, and the process may be automated. In one embodiment, the serial number of the device and the date of use may be recorded in internal memory of the console and can be made inaccessible from the user interface so as to prevent accidental erasure.

FIG. 1B shows details of the stylus in its operation position inside the needle 160 at the distal tip. The piezoelectric element 110 is oriented so that the polar axis is parallel to the long axis of both the stylus and needle. Further, the shaft 120 of the stylus is also present in the hypodermic needle 160. The piezoelectric element 110 may be positioned so that it remains behind the cutting tip 161 of the needle.

FIG. 1C illustrates some details of the stylus hub 130 connected to the needle hub 170 via a lure lock connection 130. FIG. 1D illustrates the placement of a stylus into a needle, according to an embodiment of the invention. The arrow indicates that the stylus can be introduced into the needle. FIG. 1E shows the back side of the stylus hub. FIG. 1F shows the front side of the stylus hub. The stylus position may be maintained centrally in the needle lumen to provide a passage for fluid. The arrows show direction of flow of the fluid. In one embodiment, the stylus 100 may be used to occlude the lumen opening of the needle 160 or catheter to keep it free from debris and/or add mechanical properties (rigidity of column strength) to a catheter or probe (acting like a mandrel). In an alternative embodiment, the stylus 100 may not be an active part of the medical procedure; the whole procedure could be performed without adding the piezoelectric stylus 100 into the lumen of the needle 160. Also, in an embodiment, the stylus 100 does not extend outside the confines of the needle shaft. The device may enter a needle 160, but in one embodiment may not enter the body per se.

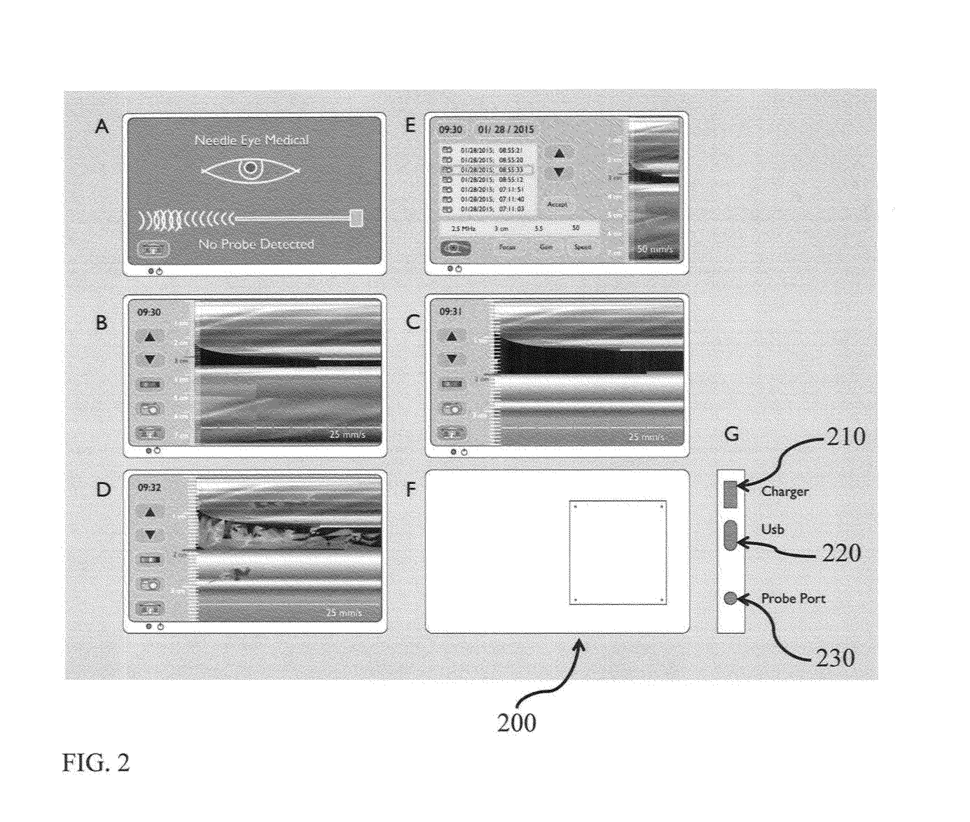

FIG. 2 illustrates a portable tablet-sized electromechanical console 200 that may be used with the stylus 100 according to an embodiment of the present invention. In one embodiment, the console 200 may be designed to withstand impact from being dropped, with certain tolerances (e.g., from 4 feet 98% of the time, etc.). The console 200 may utilize a rechargeable battery, such as with a minimize capacity (e.g., minimum 4 hours operation time, etc.). In one embodiment, the console may be water resistant and easily cleaned with standard antimicrobial disinfectant.

The console 200 may be ergonomically designed with a touch screen interface, and may have one or more stylus connection ports 230 for the stylus 100, with, for example, an RFID integrated connector. The console may also have a USB or other data port 220, a power port 210 for a power cord or charger, an on-off button, and a power indicator light (e.g., green when operational, yellow when battery life<15%, etc.).

With respect to reference A in FIG. 2, the power up screen may, according to an embodiment, have a suitable logo and a home button, and may identify the connected stylus 100 using the RFID. The device may display the connected stylus/probe 100 product name and automatically adjusts the default image settings.

With respect to reference B in FIG. 2, the screen may, according to an embodiment, switch to the default operational screen after a certain amount of time (e.g., 5-10 seconds after power on). If the home button is selected, the screen may switch to the home screen (see reference E in FIG. 2) where operational settings can be changed and data clips can be downloaded (described in further detail below).

The default operational screen may display M-mode ultrasound. The depth may be set to maximum (e.g., up to 10 cm, etc.) at default and the up and down arrows may be used to change the tissue depth (see B, C, D, in FIG. 2). The focal point (if it can be adjusted) may automatically adjust to approximately ⅓ to ½ the distance of the imaging depth.

As the imaging depth is adjusted the screen may adjust to include the range (see B in FIG. 2); the focal point may be indicated by a change in the color of the graduated markers (see A, B in FIG. 2). The sweep speed of the M-mode may be displayed and tick marks may appear at corresponding intervals (see A, B, C, D in FIG. 2). By selecting the spectrum button the color Doppler may be mapped over the over the gray scale M-mode images (see reference E in FIG. 2).

Selecting the camera button may operate to save a 5 second video (e.g., in open source file format) and a single JPEG screen image. The home screen may have user adjustments, and other suitable options may also be used. The home screen may also display an external USB device. In one embodiment, the USB port 220 may be used for exporting image data. The recorded video/image files may be named, as an example, by the date and time.

The user may select image data he/she wants to transfer to an external drive by, for example, tapping the file (it is highlighted). Tapping twice may select all of the files. If everything is selected then touching and holding in on the position for, for example, 2 seconds may select a single file. Touching and holding for 2 seconds then dragging may select adjacent files. Once the files of interest are selected the operator may tap the accept button. A sub-screen may appear that prompts the user to export to, for example, a USB port memory device and either save files on the device of the present invention, erases the files after exporting, or the like. The device memory may hold many files, or in another embodiment, only a selected number of the most recent files may be saved. In one embodiment, if, for example, only a set number of files are saved, such as 100, then the 100+1 file is saved and the oldest file (#100) may be erased.

The rear/back side of the enclosure may include a panel that can be detached with, for example, removal of fasteners. This covers the hardware maintenance access port and a secure interface for the reprogramming/updating software (see reference F of FIG. 2).

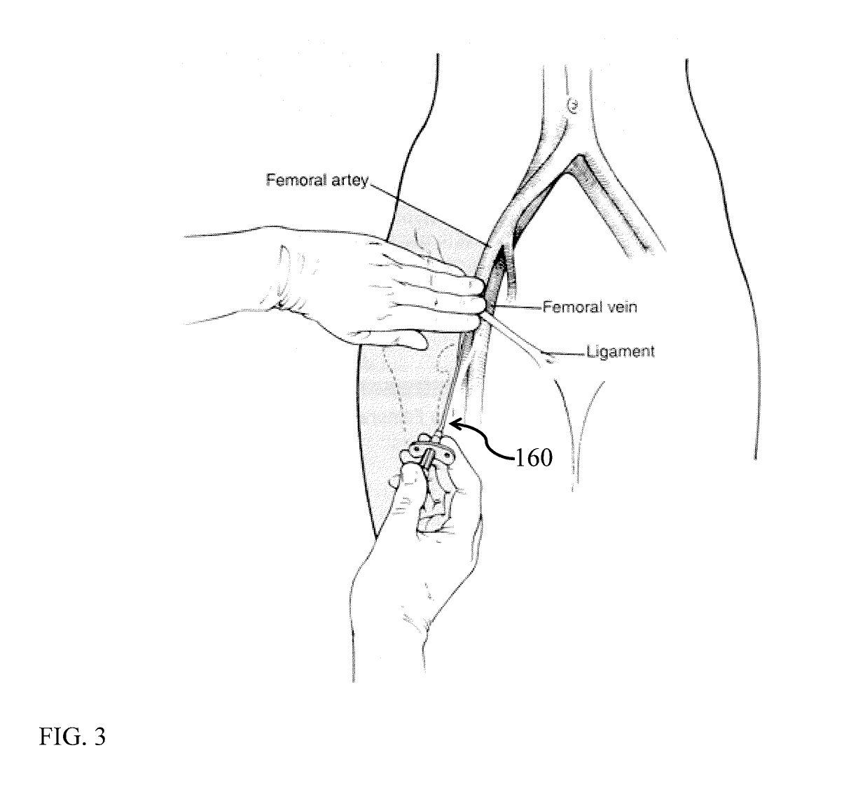

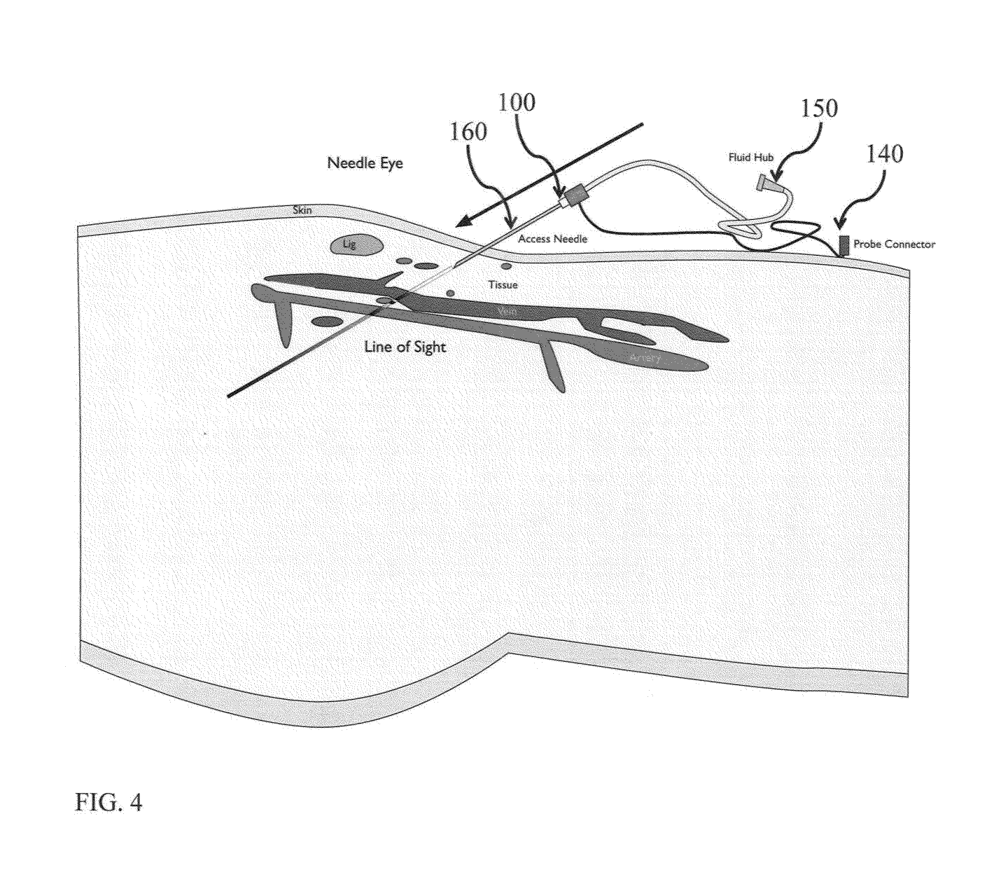

FIG. 3 and FIG. 4 illustrate the steps that may be performed to use the device according to an embodiment of the present invention. The present invention may be used with any medical procedure where a needle 160 is used. As an example, the following steps are outlined to show the operation of the device. These steps are only examples, and it will be understood that different variations may also be used.

In step 1, the operator may open the sterile packaging of the stylus 100 and insert it into the hub 170 of the needle 160 of the appropriate length. The stylus package may also each include a needle 160 that has the appropriate dimensions for optimal stylus 100 alignment. However, any needle of appropriate ID and length will work with the stylus device 100.

In step 2, the operator inserts the stylus 100 onto the back of the needle 160 so that they may twist the lure-lock 130 and verify it is secured.

In step 3, a syringe with serial saline (commonly available in all medical settings where IV medications are administered) may be used to irrigate the lumen.

In step 4, the operator attaches the external cable connector to the non-sterile extension cable (outside the sterile field).

In step 5, the stylus 100 may be connected to the console 200, a tablet sized durable medical instrument that may be operated through a touch screen interface, whose interface is described in FIG. 2. Plugging in the stylus 100 may activate the RFID, in one embodiment, and the console may display the device identification and default settings are loaded.

In step 6, the screen of the console 200 may automatically display the default M-mode image on the screen. The operator can verify function by submerging the needle/stylus tip in fluid.

In step 7, the operator may use standard anatomic landmarks and techniques to direct the needle 160. The M-mode image may be visible and can be used to adjust the needle path and avoid small vascular/anatomic structures that might cause complications.

In step 8, the image depth and/or color Doppler may be selected while performing the procedure. If the console is covered with a disposable serial sleeve then the operator can make adjustment using his or her free hand.

In step 9, the operator may choose to save an image while making the needle approach. This may, in one embodiment, be accomplished by tapping the camera icon. A 5 second video and a still shot can be saved on the console memory and indexed, for example, by date & time.

In step 10, once the needle tip is in the desired position, the stylus 100 may be removed. If further adjustment is needed the sterile stylus 100 can be reintroduced into the needle 160.

In step 11, the stylus 100 may be discarded with other soiled biohazards. In one embodiment, the RFID will not permit the stylus to be operated in the same console after a single use.

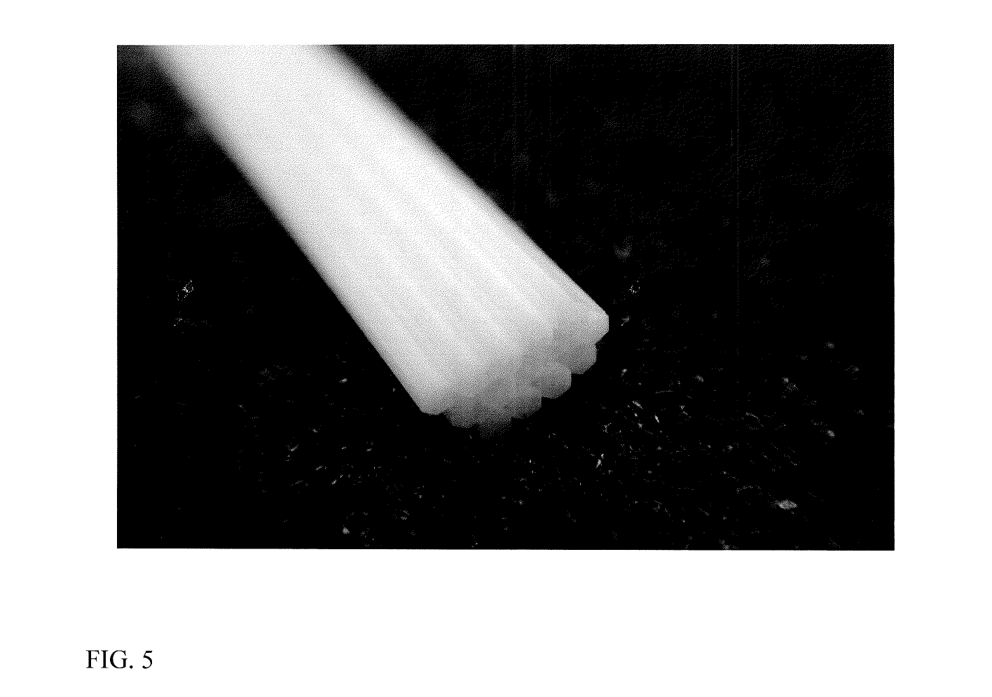

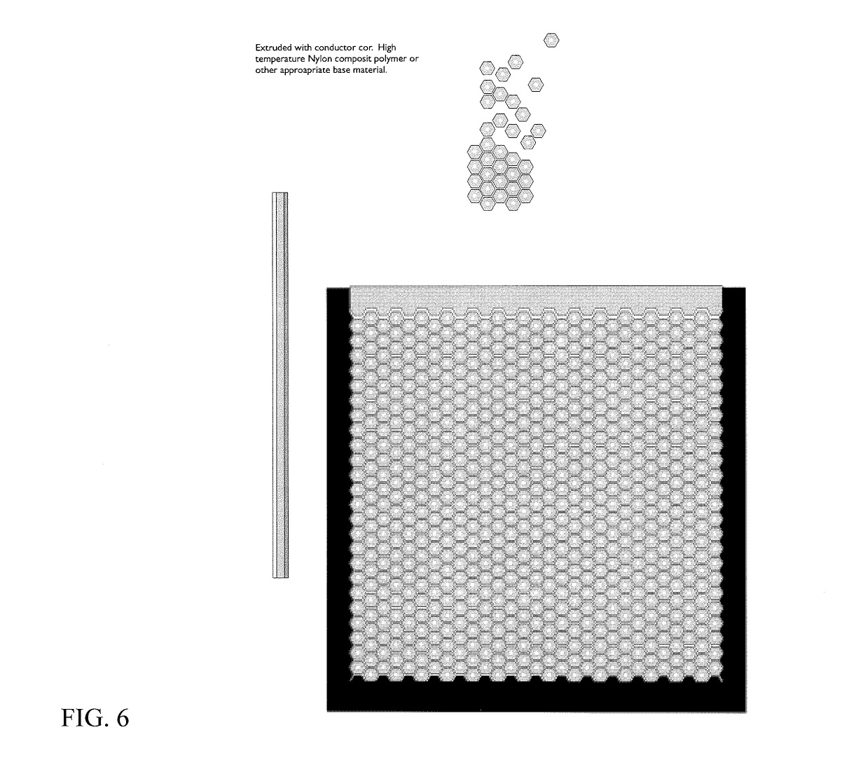

FIG. 5 shows a collection of stylus shafts according to an embodiment of the present invention. According to an embodiment of the present invention, the radial profile of the stylus shaft is a polygon that can be tessellated when multiples are grouped in parallel by long axis. In an embodiment, the stylus shaft is prepared so that the collection has an equal axial proportion and short axis plan that is perpendicular to the long axis. The planar surface of the collection of equal length tessellated styluses will have a ratio of greater than 8:2 surface to gap (excluding contributions of surface gaps resulting from gaps internal to the perimeter of the short axis). Some example shapes having this property include, but not limited to, equilateral triangle, square, rectangle, parallelogram, hexagon, and a wide array of asymmetric polygons. In an embodiment, the edges of the polygons will have a curvature when the collection is tessellated such that there is a surface to gap ratio not greater than of 8:2 (excluding any gap that results form internal to the perimeter of the short axis, such as a central lumen or tract). In an alternative embodiment, polygons with high number of facets and or curved surfaces are used. In a special circumstance where tessellation or planar alignment of stylus multiples enables manufacturing stylus with curved features such as circles and/or rounded edges may be also be used. The tessellation provides a means to manufacture the transceiver directly onto stylus en mass and provides a plane for automated application of the transceiver to multiples rather than as individuals. FIG. 6 shows the collection of styli arranged so as to show the tessellated structure. The tessellated multiples of the stylus produce a single plane that is especially practical for many automated manufacturing techniques.

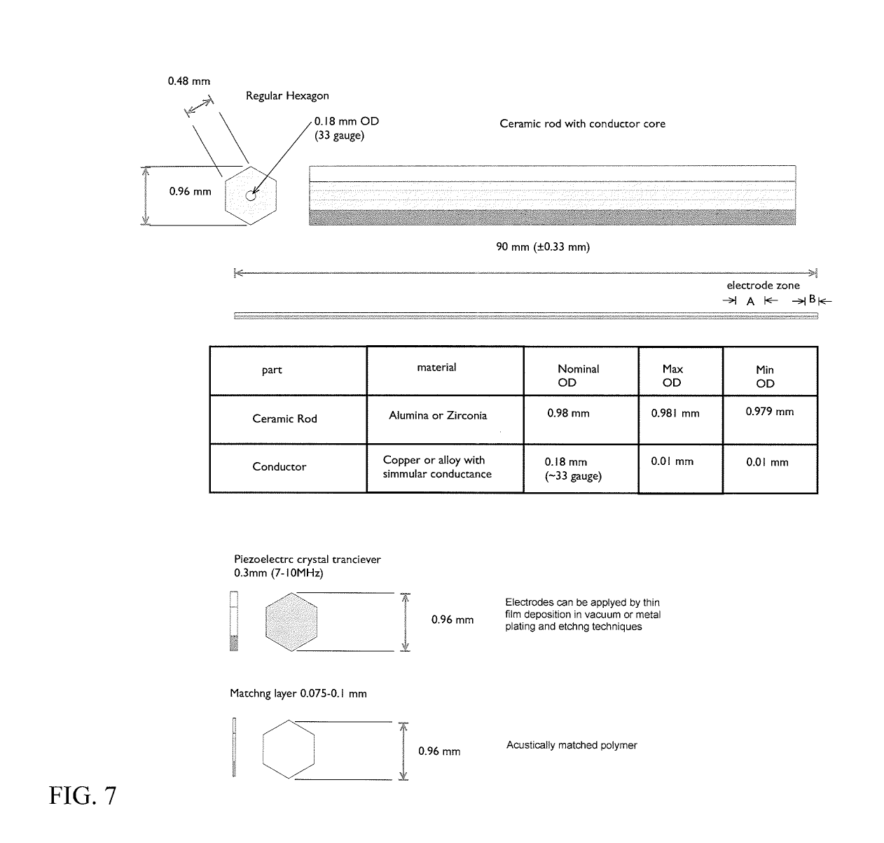

FIG. 7 illustrates the construction characteristics of a stylus. In the principal application, the transceiver will require electrodes and/or fiberoptic connections to the console in order to transmit energy from the transceiver to the signal processing component of the invention. In an embodiment, there are surface feature and/or internal lumens parallel to the long axis of the stylus that can be metalized and/or can accommodate conductors and/or fiberoptic connections. In an alternative embodiment, the transceiver connection includes an energy source or return path that is outside of the stylus, such as, but not limited tom electrical connectivity through the needle, inductive electromagnetic power source, and or return path through an external patch that would be applied to the body. The material of the stylus body may be used in whole or in part as a conductor or fiberoptic path. The principal application individual facets of the shaft have undergone a secondary processing step that provides conductive layer(s) and/or insulative layer(s) over sections of the shaft. In the primary application the shaft includes at least one metalized surface feature that is contiguous with the transceiver and proximal end of stylus. In an embodiment, the stylus includes an insulative layer that is applied over conductors following the length of the outer facet following the long axis of the shaft.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, those skilled in the art will appreciate that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. An apparatus comprising:
a piezoelectric element(s) affixed to the distal end of an elongated stylus with polar axis parallel to the long axes of the stylus and configured to be inserted into the lumen of a percutaneous needle, geometrically aligned with the distal opening.

2. The apparatus of claim 1, wherein the stylus further comprises a hub configured to be complementary to the connection of a standard percutaneous access needle.

3. The apparatus of claim 2, wherein the hub comprises one or more lure locks.

4. The apparatus of claim 3, wherein the hub is further configured to provide luminal continuity between the one or more lure locks of the hub, contiguous with the inner lumen of the percutaneous access needle, and a lure lock of the percutaneous access needle.

5. The apparatus of claim 1, further comprising a console connected to the stylus configured to regulate production of ultrasonic energy from the piezoelectric element.

6. The apparatus of claim 5, wherein the console further comprises a signal processing, analysis, and display unit for sonographic imaging comprising grey-scale M-mode images of tissue anatomy and/or color-coded Doppler maps of tissue velocity superimposed on the grey-scale image.

7. An apparatus comprising:
a piezoelectric element(s) affixed to the distal end of an elongated stylus with polar axis substantially parallel to the long axes of the stylus, the stylus configured to have a radial profile of a polygon that can be tessellated when multiples are grouped in parallel by the long axis.

8. The apparatus of claim 7, wherein the stylus has an equal axis proportion and short axis plan that is perpendicular to the long axis.

9. The apparatus of claim 7, wherein a planar surface of the stylus, when gathered in a collection, has a surface to gap ratio of 8 to 2, exclusive of contributions of surface gaps resulting from gaps internal to the perimeter of a short axis of the stylus.

10. The apparatus of claim 9, wherein the planar surface of the stylus comprises a triangle shape.

11. The apparatus of claim 9, wherein the planar surface of the stylus comprises a hexagonal shape.

12. The apparatus of claim 9, wherein the planar surface of the stylus comprises a parallelogram shape.

13. The apparatus of claim 7, wherein the stylus comprises a shaft, the shaft comprising a conductor.

14. The apparatus of claim 13, wherein the shaft further comprises an insulating layer.

15. The apparatus of claim 7, wherein the stylus comprises a shaft, the shaft comprising a fiber optic path.

16. An method of using a front facing line-of-sight stylus probe to assist needle guidance comprising:
Inserting a stylus comprising a piezoelectric element tip into a needle having a distal needle opening;
sending and measuring the sonographic energy from the piezoelectric element configured so that the polar axis is parallel to the long axes of the stylus and the element is aligned/oriented closely with the distal opening of the needle so that the ultrasound is emanating and measured from the distal needle opening.

17. The method of claim 16, further comprising injecting a fluid through a lumen comprising a hub on the stylus hub, flowing the fluid around the stylus, and exiting the fluid from the distal opening of the needle.

18. The method of claim 16, further comprising aspirating a fluid through a lumen comprising a hub on the stylus, flowing the fluid around a shaft of the stylus occupying a portion of an inner lumen of the needle, and exiting the fuid from the distal opening to the stylus hub and needle.

19. The method of claim 16, further comprising advancing the needle and the stylus percutaneously through soft tissue with simultaneous acquisition of an ultrasound imaging and/or Doppler data for the purpose of performing a medical procedure.

20. The method of claim 16, further comprising adjusting the image focal depth of the stylus and adding color Doppler mapping for the purpose of resolving tissues structure details over a range of distances and identifying vascular structures, collateral anatomy, or target tissues in the line-of-sight and path of the needle.
